(19) United States
(12) Patent Application Publication
(10) Pub. No.: US 2022/0401131 A1
(43) Pub. Date: Dec. 22, 2022

(54) Spinal Rod Preparation Systems and Methods

(71) Applicant: Trailhead Innovations, LLC, Irvine, CA (US)

(72) Inventors: Nicholas Hu, Irvine, CA (US); Kevin Hughes, Irvine, CA (US); Jason Liauw, Irvine, CA (US); Lauren Lee, Irvine, CA (US)

(21) Appl. No.: 17/774,441

(22) PCT Filed: Nov. 5, 2020
(86) PCT No.: PCT/US2020/059103

§ 371 Date: May 4, 2022

Related U.S. Application Data
(60) Provisional application No. 62/930,969, filed on Nov. 5, 2019.

(51) Int. Cl. A61B 17/70; A61B 17/88; A61B 90/00; A61B 34/10

(52) U.S. Cl. CPC A61B 17/7013; A61B 17/8863; A61B 90/361; A61B 34/10; A61B 2034/102; A61B 2017/00946

(57) ABSTRACT

The present disclosure provides systems and methods for preparing a spinal rod that enable the digital mapping of rod contours to produce spinal rods that conform to an ideal rod trajectory, which reduces spinal rod to screw head misalignment. Reducing spinal rod to screw head misalignment helps reduce a failure rate of spinal rods in patients. In invasive spinal fusion surgeries, a digital three-dimensional representation may be generated of a flexible rod formed to align with screws installed in the patient. In minimally invasive surgeries, a digital three-dimensional representation may be generated using pointers. A surgeon may adjust the digital three-dimensional representation via a graphical user interface. Bending instructions may be generated from the digital three-dimensional representation that direct how a spinal rod should be bent using a bending tool. The final spinal rod accounts for the anatomical environment around the screws installed in the patient.

### PRIORITY CLAIM

The present application claims priority to and the benefit of U.S. Provisional Application 62/930,969, filed Nov. 5, 2019, the entirety of which is herein incorporated by reference.

### BACKGROUND

During spinal fusion surgeries, spinal rod constructs must be formed intraoperatively to align with screw positions and intended patient spinal curvature. The standard of care approach for doing so requires that the surgeon bend spinal rods by eye using handheld instruments. This standard of care method proves to be both time-consuming and burdensome for a surgeon, and can lead to excessive reduction on screws in order to align them to the hand bent rod. For instance, a limitation of such typical methods lies in the operator induced error observed, since the surgeon is tasked with mapping and planning everything by eye, with no access to assistive technology. The resulting excessive reduction on screws can introduce undesired stresses on the installed spinal rod and/or screws, which can lead to an undesired incidence of failure of the installed spinal rod and/or screws.

Other typical methods have attempted to address the limitations introduced by the standard of care approach by aiming to streamline the workflow for generating spinal rod constructs via the introduction of assistive technology (e.g., U.S. Pat. No. 9,636,181). However, these attempts are also met with limitations and leave room for improvement. Such assistive technology determines the location of spinal screws, along patient spinal anatomy, and relevant fixation members to identify points that are then used to calculate a curve. The calculated curve is then translated into a set of bending instructions to be utilized by the operator when generating spinal rod constructs. This approach, though an improvement from the standard of care, is restricted to chosen screw points, with no consideration for the contour of the surrounding tissue environment. This can create less than ideal rod contours that can run into tissue interference and can create less than ideal approach trajectories to screws, which can place a lot of angular strain on screws on final tightening.

Accordingly, systems and methods that address the above drawbacks for preparing a spinal rod construct are desired.

### SUMMARY

The present disclosure provides new and innovative systems and methods for intraoperatively preparing a spinal rod for invasive and minimally invasive spinal fusion surgeries.

In light of the disclosures herein, and without limiting the scope of the invention in any way, in a first aspect of the present disclosure, which may be combined with any other aspect listed herein unless specified otherwise, a spinal rod preparation system includes a flexible rod, a bending instructions generation subsystem, and/or a bending tool. The flexible rod is configured to be manipulated to a bent state. The bending instructions generation subsystem includes an image capture device and a processor in communication with a memory. The processor is configured to receive at least two images of the flexible rod in the bent state, captured by the image capture device. A digital three-dimensional representation of the flexible rod in the bent state is generated based on the at least two received images. Adjustment information is received that adjust the digital three-dimensional representation of the flexible rod to an adjusted shape. The processor may generate bending instructions for bending a spinal rod so that it substantially conforms to the adjusted shape of the digital three-dimensional representation. The bending tool may be configured to enable a user to bend a straight spinal rod according to the generated bending instructions.

In a second aspect of the present disclosure, which may be combined with any other aspect listed herein unless specified otherwise, the spinal rod preparation system further includes a fixture configured to maintain a positioning of the flexible rod while the image capture device captures the at least two images of the flexible rod in the bent state.

In a third aspect of the present disclosure, which may be combined with any other aspect listed herein unless specified otherwise, the flexible rod extends within a plane perpendicular to a base of the fixture when maintained by the fixture.

In a fourth aspect of the present disclosure, which may be combined with any other aspect listed herein unless specified otherwise, the fixture includes a fiducial marker, wherein the fiducial marker is captured in the at least two images of the flexible rod.

In a fifth aspect of the present disclosure, which may be combined with any other aspect listed herein unless specified otherwise, the flexible rod is constructed of a malleable material.

In a sixth aspect of the present disclosure, which may be combined with any other aspect listed herein unless specified otherwise, the flexible rod includes a plurality of articulating joints.

In a seventh aspect of the present disclosure, which may be combined with any other aspect listed herein unless specified otherwise, the flexible rod is constructed of a material capable of transitioning from malleable to rigid upon a transitioning event.

In an eighth aspect of the present disclosure, which may be combined with any other aspect listed herein unless specified otherwise, the processor is configured to generate the digital three-dimensional representation based on three or more images via a photogrammetry method.

In a ninth aspect of the present disclosure, which may be combined with any other aspect listed herein unless specified otherwise, the image capture device is configured to capture video, and the processor is configured to generate the digital three-dimensional representation based on a captured video of the flexible rod in the bent state.

In a tenth aspect of the present disclosure, which may be combined with any other aspect listed herein unless specified otherwise, a spinal rod preparation method includes aligning a flexible rod with a plurality of screws installed in a patient. Aligning the flexible rod includes bending the flexible rod to conform to a path of the plurality of screws. At least two images of the aligned flexible rod may be captured via an image capture device. A digital three-dimensional representation is generated of the aligned flexible rod via a bending instructions generation subsystem based on the at least two captured images. The digital three-dimensional representation of the aligned flexible rod is adjusted via a user interface of the bending instructions generation subsystem. A spinal rod may be bent using a bending tool according to bending instructions generated by the bending instructions generation subsystem based on the adjusted digital three-dimensional representation.

In an eleventh aspect of the present disclosure, which may be combined with any other aspect listed herein unless specified otherwise, the images are captured while the flexible rod is positioned within heads of the plurality of screws.

In a twelfth aspect of the present disclosure, which may be combined with any other aspect listed herein unless specified otherwise, the images are captured using a fixture away from the plurality of screws.

In a thirteenth aspect of the present disclosure, which may be combined with any other aspect listed herein unless specified otherwise, capturing the at least two images includes capturing a first image of the aligned flexible rod in a first position and a second image of the aligned flexible rod in a second position, and wherein the first position is orthogonal to the second position.

In a fourteenth aspect of the present disclosure, which may be combined with any other aspect listed herein unless specified otherwise, adjusting the digital three-dimensional representation includes interacting with a graphical user interface including the digital three-dimensional representation.

In a fifteenth aspect of the present disclosure, which may be combined with any other aspect listed herein unless specified otherwise, adjusting the digital three-dimensional representation includes selecting one or more points on the digital three-dimensional representation and adjusting an amount of curvature at or between the one or more points.

In a sixteenth aspect of the present disclosure, which may be combined with any other aspect listed herein unless specified otherwise, a spinal rod preparation system includes a scan rod, a plurality of pointers, a bending instructions generation subsystem, and/or a bending tool. The pointers each have at least two fiducial markers connected to one another by a shaft, and each are configured to attach to the scan rod. The bending instructions generation subsystem includes an image capture device and a processor in communication with a memory. The processor is configured to receive at least two images of the plurality of pointers attached to the scan rod, captured by the image capture device. A digital three-dimensional representation of a spinal rod is generated based on the at least two received images. Adjustment information is received that adjust the digital three-dimensional representation to an adjusted shape. The processor may generate bending instructions for bending a spinal rod so that it substantially conforms to the adjusted shape of the digital three-dimensional representation. The bending tool may be configured to enable a user to bend a straight spinal rod according to the generated bending instructions.

In a seventeenth aspect of the present disclosure, which may be combined with any other aspect listed herein unless specified otherwise, generating the digital three-dimensional representation of the spinal rod includes determining a vector of a direction in which a pointer is directed based on the at least two fiducial markers of the pointer and generating the digital three-dimensional representation based on the determined vectors for each of the plurality of pointers.

In an eighteenth aspect of the present disclosure, which may be combined with any other aspect listed herein unless specified otherwise, the spinal rod preparation system further includes a fixture configured to maintain a positioning of the scan rod while the image capture device captures the at least two images of the plurality of pointers attached to the scan rod.

In a nineteenth aspect of the present disclosure, which may be combined with any other aspect listed herein unless specified otherwise, each pointer is configured to be inserted partially within a MIS tower installed in a patient.

In a twentieth aspect of the present disclosure, which may be combined with any other aspect listed herein unless specified otherwise, the bending tool is configured to adjust a positioning of a coupled spinal rod both translationally and axially, and to adjust a bend angle that the bending tool effects upon the coupled spinal rod in response to an arm of the bending tool being actuated.

In a twenty-first aspect of the present disclosure, which may be combined with any other aspect listed herein unless specified otherwise, the bending instructions include at least one set of instructions for at least one bend, each set of instructions including a set translational position for the coupled spinal rod, a set rotational position for the coupled spinal rod, and a set bend angle.

In a twenty-second aspect of the present disclosure, which may be combined with any other aspect listed herein unless specified otherwise, a spinal rod preparation method includes positioning a plurality of pointers into respective MIS towers installed in a patient. Each pointer has at least two fiducial markers connected to one another by a shaft. The plurality of pointers may be attached to a scan rod. At least two images of the plurality of pointers attached to the scan rod may be captured via an image capture device. A digital three-dimensional representation of a spinal rod may be generated via a bending instructions generation subsystem based on the at least two captured images. The digital three-dimensional representation of the spinal rod may be adjusted via a user interface of the bending instructions generation subsystem. A spinal rod may be bent using a bending tool according to bending instructions generated by the bending instructions generation subsystem based on the adjusted digital three-dimensional representation.

In a twenty-third aspect of the present disclosure, which may be combined with any other aspect listed herein unless specified otherwise, the spinal rod is coupled to the bending tool while bending the spinal rod, and wherein bending the spinal rod according to the bending instructions includes adjusting the bending tool so that the coupled spinal rod is at a first translational position and a first rotational position. The bending tool may be adjusted to a first set bend angle. An arm of the bending tool may be actuated.

In a twenty-fourth aspect of the present disclosure, which may be combined with any other aspect (e.g., the twenty-third) listed herein unless specified otherwise, bending the spinal rod according to the bending instructions includes adjusting the bending tool so that the coupled spinal rod is at a second translational position and a second rotational position. The second translational position is different than a first translational position. The bending tool may be adjusted to a second set bend angle. The arm of the bending tool may be actuated.

### BRIEF DESCRIPTION OF THE DRAWINGS

FIG. 1 illustrates a spinal rod preparation system, according to an aspect of the present disclosure.

FIG. 2 illustrates a portion of a patient's vertebral column having installed screws, according to an aspect of the present disclosure.

FIG. 3 illustrates a shaped flexible rod, according to an aspect of the present disclosure.

FIG. 4 illustrates a perspective view of an example fixture, according to an aspect of the present disclosure.

FIG. 5 illustrates a captured image of a shaped flexible rod mated to a fixture in a first position, according to an aspect of the present disclosure.

FIG. 6 illustrates a captured image of a shaped flexible rod mated to a fixture in a second position with respect to the first position illustrated in FIG. 5, according to an aspect of the present disclosure.

FIG. 7 illustrates a graphical user interface including a digital three-dimensional representation of an intended spinal rod, according to an aspect of the present disclosure.

FIG. 8 illustrates a graphical user interface including bending instructions, according to an aspect of the present disclosure.

FIG. 9A illustrates a perspective view of a bending tool, according to an aspect of the present disclosure.

FIG. 9B illustrates a top view of the bending tool of FIG. 9A, according to an aspect of the present disclosure.

FIG. 9C illustrates a perspective view of the bending tool of FIG. 9A having a loaded spinal rod, according to an aspect of the present disclosure.

FIG. 10 illustrates a patient having installed MIS towers, according to an aspect of the present disclosure.

FIG. 11 illustrates a perspective view of a pointer, according to an aspect of the present disclosure.

FIG. 12 illustrates a perspective view of a scan rod, according to an aspect of the present disclosure.

FIG. 13 illustrates a perspective view of a set of pointers both inserted into the MIS towers installed in the patient of FIG. 10 and attached to a scan rod, according to an aspect of the present disclosure.

FIG. 14 illustrates a captured image of a scan rod, including attached pointers, mated to a fixture, according to an aspect of the present disclosure.

### DETAILED DESCRIPTION

The present disclosure provides systems and methods for preparing a spinal rod that enable the digital mapping of rod contours to produce spinal rods that conform to an ideal rod trajectory, which reduces spinal rod to screw head misalignment. Reducing spinal rod to screw head misalignment helps reduce a failure rate of spinal rods in patients. The provided system not only introduces assistive technology (e.g., a computing system) to streamline spinal rod construct fabrication as compared to the standard of care method, but also accounts for the surrounding environment, without being tied down by the vertebrae and attachments points alone as compared to other typical methods. With technology that accounts for anatomical interference and that can accurately represent patient spinal contour, the provided system enables preparing spinal rods with a higher resolution than typical methods. The implementation of assistive technology that accounts for surrounding anatomical structures and that maintains accuracy of the patient spinal contour helps to automate planning and execution of spinal rod preparation, which helps reduce at least some operator induced error. The provided systems and methods will also reduce surgical time, improving the quality of care for the patient, by helping a surgeon prepare a spinal rod correctly the first time, thus avoiding repeated manual adjustments.

Preparing a spinal rod may involve an invasive procedure or a minimally invasive procedure. Depending on the procedure, the presently disclosed system provides components for creating a digital three-dimensional representation of a spinal rod and generating instructions for bending a spinal rod to substantially conform to the digital three-dimensional representation. For an invasive procedure, a surgeon may bend a flexible rod to align with screws installed in a patient. The flexible rod is used as a reference model of a spinal rod for image generation and is more flexible than a spinal rod that will ultimately be implanted. The surgeon may then capture images or video of the aligned flexible rod with a camera. In some aspects, the images or video may be captured in situ while the flexible rod remains at or within the patient. In other aspects, the aligned flexible rod may be transferred to a fixture that maintains various positioning of the aligned flexible rod for image or video capture.

A computing system (e.g., a tablet computer) may then analyze the images or video to generate a digital three-dimensional representation of the aligned flexible rod, which may be referred to as a representation of an intended spinal rod. Generating the digital three-dimensional representation based on a physical reference structure that models an intended spinal rod construct helps account for the anatomical environment in which the final spinal rod construct will be installed, without being tied down by the vertebrae and installed screw positioning (e.g., attachments points) alone as compared to other typical methods. Accounting for the anatomical environment helps prepare a spinal rod that minimizes undesired stresses on the spinal rod and the screws when the spinal rod is installed, which helps minimize undesired failure of the spinal rod and/or screws.

The digital three-dimensional representation may be presented on a display of the computing system as a graphical user interface that enables the surgeon to interact with and adjust the digital three-dimensional representation. For example, the surgeon may select two points on the digital three-dimensional representation and adjust an amount of bend between those two points. Once the surgeon is satisfied with the shape of the digital three-dimensional representation, the surgeon may initiate bending instructions generation on the computing system. The computing system generates instructions for bending a straight spinal rod so that it substantially conforms to the digital three-dimensional representation. For example, the bend instructions may be generated to minimize a quantity of bends needed for the straight spinal rod so that it best conforms with the digital three-dimensional representation. In such examples, the final spinal rod after bending might not exactly match the digital three-dimensional representation and/or the flexible rod, but has close conformance (e.g., substantially conforms).

In some aspects, the surgeon may bend a straight spinal rod using a bending tool. In such aspects, the bending instructions generated by the computing system may be tailored to the particular bending tool used. In one example, a spinal rod may couple to a bending tool that includes three degrees of adjustment for bending—translational position of the spinal rod, axial rotation of the spinal rod, and a set bend angle. In such an example, the generated bending instructions may include an instruction or value for each of these three degrees of adjustment for one or more bends to be performed. After the surgeon bends a spinal rod according to the bending instructions, the spinal rod is prepared and ready for installation in the patient.

In a minimally invasive procedure, multiple minimally invasive surgery (MIS) towers are positioned such that they contact the screws installed in the patient and extend exteriorly to the patient's skin. For these procedures, the surgeon does not have access into the patient to align a flexible rod with the installed screws. Instead, the surgeon may utilize multiple pointers that capture points related to an intended spinal rod structure. A pointer may be positioned within each MIS tower and without contacting the installed screws. In an example, each pointer may include at least two fiducial markers connected to one another by a straight shaft. In at least some aspects, the surgeon may attach each of the positioned pointers to a scan rod that acts as an anchor or guide for the pointers. The surgeon may then capture images or video of the pointers attached to the scan rod with a camera. In some aspects, the images or video may be captured in situ while the attached pointers remain at or within the MIS towers. In other aspects, the pointers attached to the scan rod may be transferred to a fixture that maintains various positioning of the pointers attached to the scan rod for image or video capture.

A computing system (e.g., a tablet computer) may then analyze the images or video to generate a digital three-dimensional representation of a spinal rod. In at least some aspects, the digital three-dimensional representation may be generated based on vectors that are determined from the fiducial markers of each of the pointers. Similar to the invasive procedure, in various aspects, the digital three-dimensional representation may be presented as a graphical user interface and/or the computing system may generate bending instructions. The final spinal rod construct in the minimally invasive procedure may be prepared according to the bending instructions in the same manner described for the invasive procedure.

The pointers account for surrounding anatomical structures by following trajectories provided by the MIS towers. The pointers help determine locations of the bottoms of the MIS towers (e.g., where the MIS towers contact the screws), and therefore the pointers account for anatomy because the MIS towers' purpose is to provide access to the anatomy, which indirectly accounts for anatomy. Furthermore, an additional advantage of the pointers versus typical systems is that multiple trajectories of each of the pointers are captured all at once, capturing the contour of multiple areas of the spinal column at once. Conversely, atypical system captures spinal contour at different areas one by one. The pointers with the scan rod give the surgeon a tangible, visual representation of the captured spinal contour, allowing the surgeon a visual confirmation of the intended spinal rods. The surgeon can, if need be, tweak the geometry captured by the pointers.

Additional advantages of the provided spinal rod preparation systems and methods will be apparent from the following description of the figures.

Throughout the disclosure, reference is made to a surgeon. It should be appreciated that a surgeon may alternatively be any other suitable healthcare professional or other suitable user of the provided spinal rod preparation systems and methods.

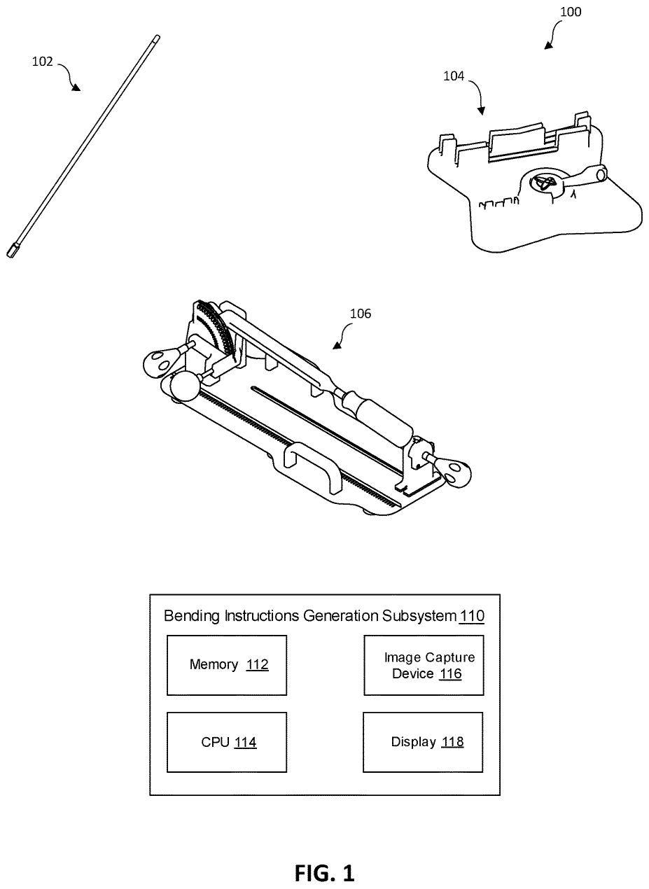

FIG. 1 illustrates an example spinal rod preparation system 100. In at least some aspects, the system 100 includes a flexible rod 102. Because the flexible rod 102 may be positioned within a patient, it is constructed of a biocompatible material. The flexible rod 102 is malleable or adjustable such that it can be shaped into any desired contour and maintains its shape once adjusted. For example, in some aspects of the present disclosure the flexible rod 102 may be a wire constructed of a malleable material (e.g., aluminum, stainless steel, cobalt chromium alloys). In some instances, the malleable wire may be covered by a sheath, such as a plastic sheath (e.g., a thermoplastic or silicone). In another example, the flexible rod 102 may include a series of rigid structures connected by articulating joints. In such an example, the articulating joints enable the flexible rod 102 to be shaped into a desired contour and maintain that desired contour. The articulating joints can be constructed from a variety of suitable materials, such as plastics spheres, polymers, moldable clay, or other suitable materials.

In some aspects, the flexible rod 102 may be constructed of a material that undergoes a transition from a malleable state to a rigid state. Implementing a suitable material that transitions from malleable to rigid helps minimize or eliminate any change in shape or spring-back that might occur (e.g., inadvertently or due to material properties) after the flexible rod 102 is shaped to a desired contour. Such change in shape or spring back would, in at least some instances, reduce the accuracy with which a final spinal rod construct is prepared for a patient. In such aspects, the flexible rod 102 is used in its malleable state to capture patient spinal contour, and is then removed from the patient for exposure to a secondary process that induces a transition from malleable to rigid.

In an example of such aspects, the flexible rod 102 may be constructed as a tube filled with an ultraviolet (UV) cured glue or other suitable UV-cured substance that transitions from malleable to rigid when exposed to UV curing. In another example, the flexible rod 102 may be constructed as a tube filled with sand or beat particles that when exposed to a vacuum, compress, and form a rigid structure due to experienced friction against tube walls. In another example still, the flexible rod 102 may be constructed as a tube filled with a liquid mixture that is induced to experience a phase change upon freezing (e.g., crystallization). For instance, one example utilizes a supersaturated sodium sulfate solution that is catalyzed to crystallize, thus forming a rigid structure. In a further example, the flexible rod may be constructed to include an expanding/hardening foam.

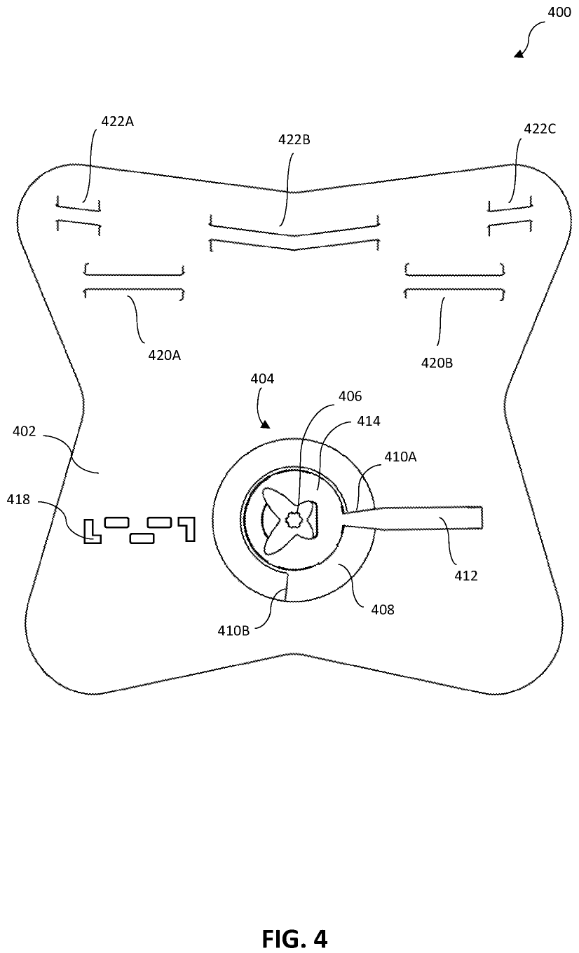

In some aspects, the system 100 may include a fixture 104 that helps maintain a positioning of the flexible rod 102. For instance, in some aspects, the fixture 104 may maintain various positioning of the flexible rod 102 during image or video capture. In some instances, the flexible rod 102 may be mated to a fixture 104 that includes discrete, fixed positions for the mated flexible rod 102 (e.g., at least two positions that are a rotation from one another). An example of such a fixture 104 is presented in FIG. 4 described below. In other aspects, image or video may be captured of the flexible rod 102 without the use of the fixture 104. For example, image or video may be captured while the flexible rod 102 remains within the patient. In another example, a variation of a contour ruler may be utilized to capture spinal contour by tracing the geometry of the contour onto suitable material having indication points to designate a position. In such examples, an image may be captured of the traced image including the indication points.

In at least some aspects, the system 100 includes a bending instructions generation subsystem 110. In an example, the bending instructions generation subsystem 110 may be a tablet computer or other suitable computing device. The bending instructions generation subsystem 110 includes a memory 112. The memory 112 may be in communication with a processor. The processor may be a CPU 114, an ASIC, or any other similar device. The bending instructions generation subsystem 110 may be configured to capture images or video in a variety of suitable manners in various aspects, such as optical imaging, lasers, or digitized gauges, with an image capture device 116. The image capture device 116 may be any suitable device capable of capturing images and/or video, such as a camera. It should be appreciated that video may be a series of still images in rapid succession.

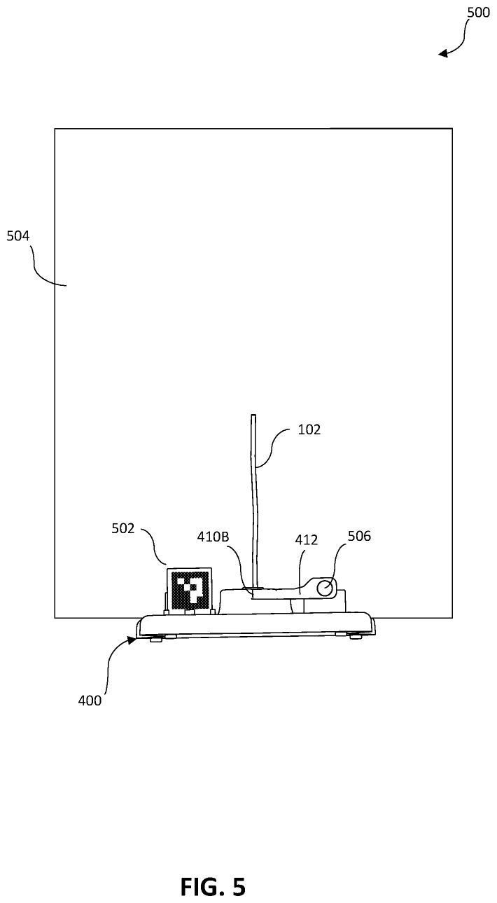

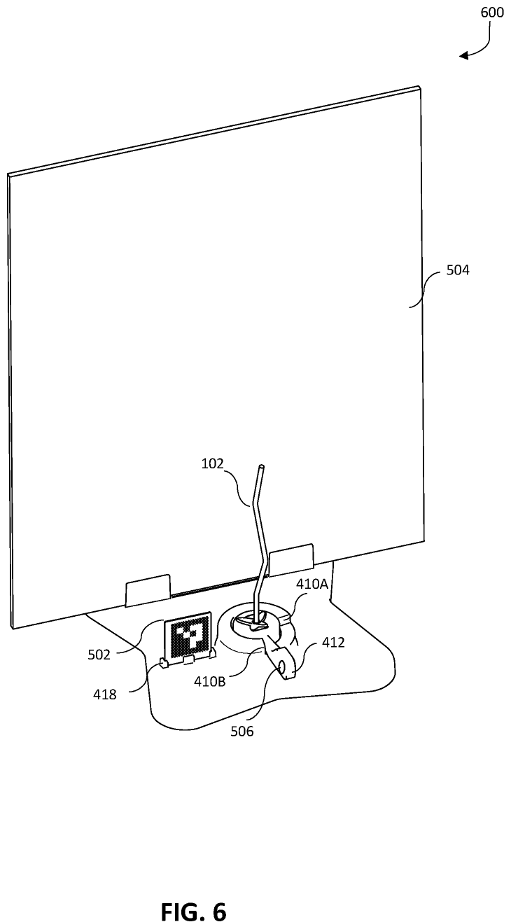

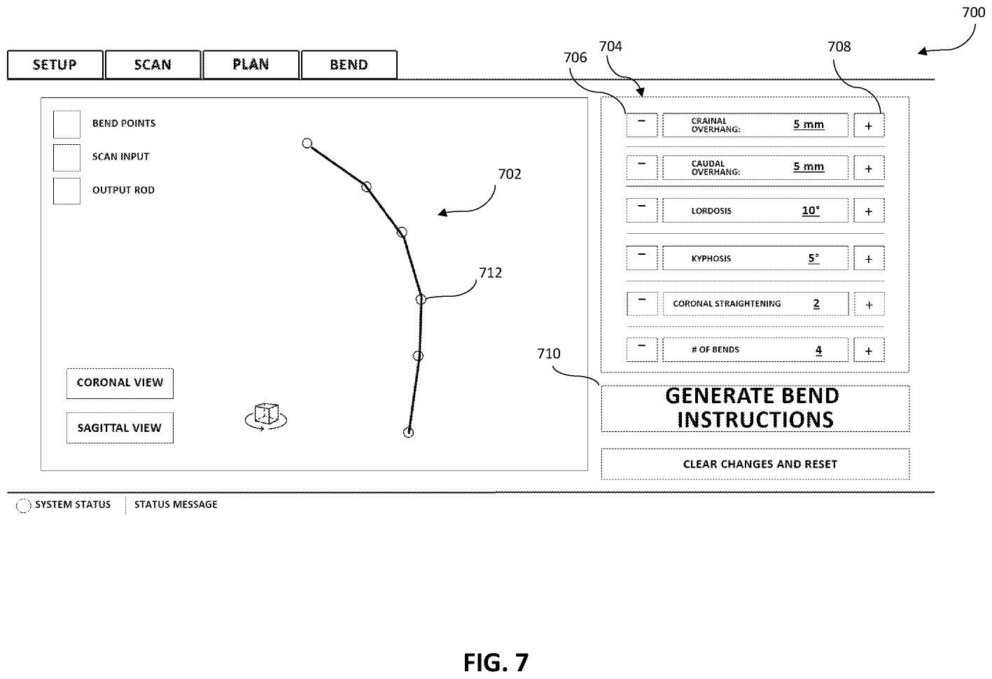

The bending instructions generation subsystem 110 may generate a digital three-dimensional representation 702 (FIG. 7) of the flexible rod 102 based on the captured images and/or video of the flexible rod 102. This generated digital three-dimensional representation 702 of the flexible rod 102 may be referred to as a digital three-dimensional representation 702 of an intended spinal rod. In some aspects, the bending instructions generation subsystem 110 may generate the digital three-dimensional representation 702 based on two images of the flexible rod 102 that are captured at an angle (e.g., ninety degrees) from one another (e.g., FIGS. 5 and 6). In some aspects, the two images may be captured at any suitable known angle from one another. In other aspects, the angle need not be known between the two captured images if a fiducial card (e.g., fiducial card 502 in FIG. 5) is rotated with the flexible rod 102.

The two images enable the bending instructions generation subsystem 110 to execute digital three-dimensional reconstruction of the flexible rod 102 captured in the two images via at least one known constant between the two images. In some instances, a known constant may be a numerical input, such as a length or diameter of the flexible rod 102. In some instances, a known constant may be an environmental factor, such as a position of a fiducial marker. As compared to typical three-dimensional reconstruction systems or methods, the bending instructions generation subsystem 110 enables generating a three-dimensional reconstruction of the flexible rod 102 via rotating the flexible rod 102 rather than rotating the image capture device. In at least some instances, it is advantageous to maintain the image capture device in a consistent position during a spinal fusion surgical procedure.

In other aspects, the bending instructions generation subsystem 110 may generate the digital three-dimensional representation 702 based on two or more images of the flexible rod 102 irrespective of the angles at which the two images were captured. In such other aspects, the bending instructions generation subsystem 110 may utilize a known photogrammetry method, such as a known triangulation technique, to generate the digital three-dimensional representation 702. As will be appreciated, triangulation refers to the process of determining a point in three-dimensional space given its projections onto two, or more, images. Triangulation requires solving the correspondence problem in order to best filter points taken from multiple images. In some aspects, to solve the correspondence problem, the bending instructions generation subsystem 110 may execute at least one of: (1) path tracing to ensure the order of discrete locations on the captured flexible rod 102 in the images, (2) iterative estimation of correspondence and re-projection based on two known planes or projections, and (3) midpoint selection and assumptions made after image correction, where image correction addresses issues such as length distortion.

In other aspects still, the bending instructions generation subsystem 110 may generate the digital three-dimensional representation 702 based on a video (e.g., three-dimensional scan) captured of the flexible rod 102. For example, the bending instructions generation subsystem 110 may generate the digital three-dimensional representation 702 via point cloud three-dimensional scanning. In some instances, point clouds can be generated utilizing a laser-based system that acts as a reading system, generating points that represent patient spinal contour. In at least some instances, generating the digital three-dimensional representation 702 based on video results in greater overall accuracy than generating it based on two or more images.

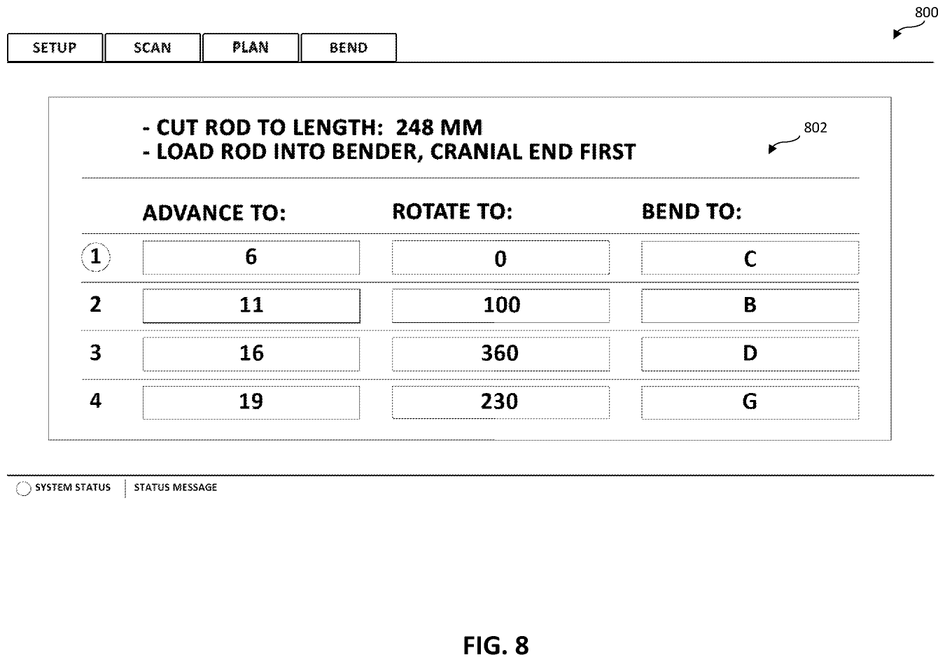

The generated digital three-dimensional representation 702 may be displayed as part of a graphical user interface (GUI) on a display 118 of the bending instructions generation subsystem 110. In some aspects, the display 118 may be a touchscreen that enables a surgeon to interact with the GUI (e.g., with the digital three-dimensional representation 702) by touching, or executing various motions (e.g., two finger slide or rotation) on the display 118. For example, the surgeon may adjust a shape of, or rotate, the digital three-dimensional representation 702 by directly interacting with the display 118. Such adjustments to the digital three-dimensional representation 702 will be described in connection with FIG. 7 below. The bending instructions generation subsystem 110 may generate bending instructions 802 (FIG. 8) based on the digital three-dimensional representation 702. In other examples, the components of the bending instructions generation subsystem 110 may be combined, rearranged, removed, or provided on a separate device or server.

In at least some aspects, the system 100 includes a bending tool 106. In some aspects, the bending tool 106 is configured to help enable a surgeon to manually bend a spinal rod according to the bending instructions 802 generated by the bending instructions generation subsystem 110. For instance, the bending tool 106 may be a handheld bending tool. In such instances, the spinal rod can include markings or indentations to indicate axial rotational positions and translational positions along the spinal rod's length at which the spinal rod should be bent with the handheld bending tool. The handheld bending tool may include corresponding indications for how it should be positioned relative to the spinal rod. A surgeon can position the spinal rod relative to the handheld bending tool and execute a bend with the tool.

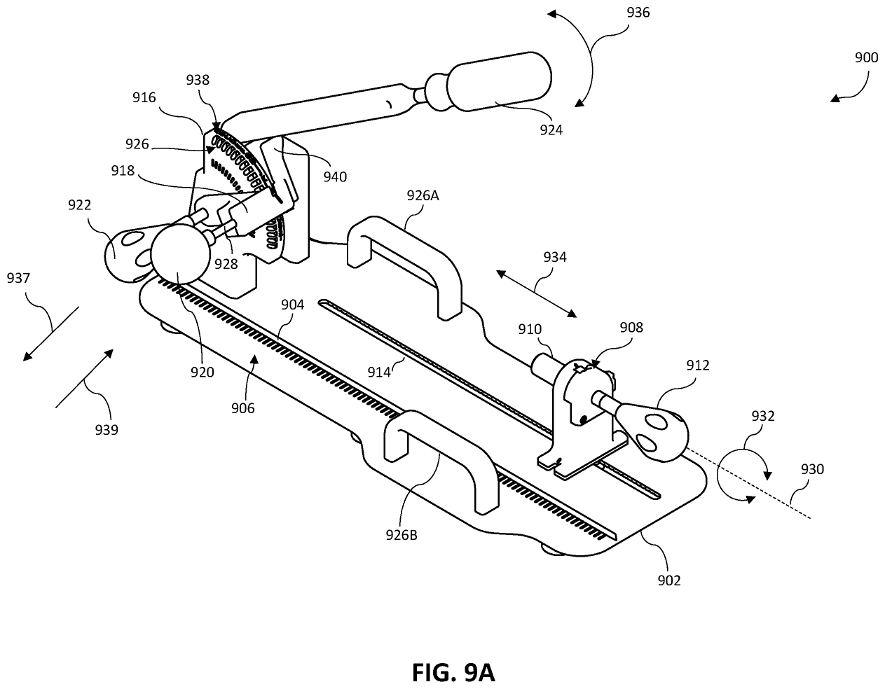

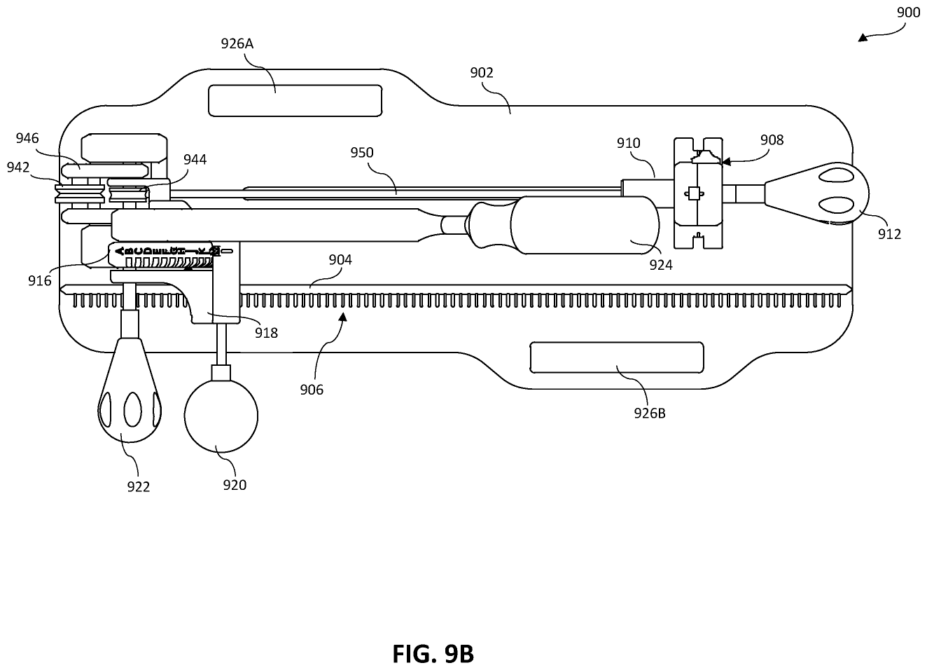

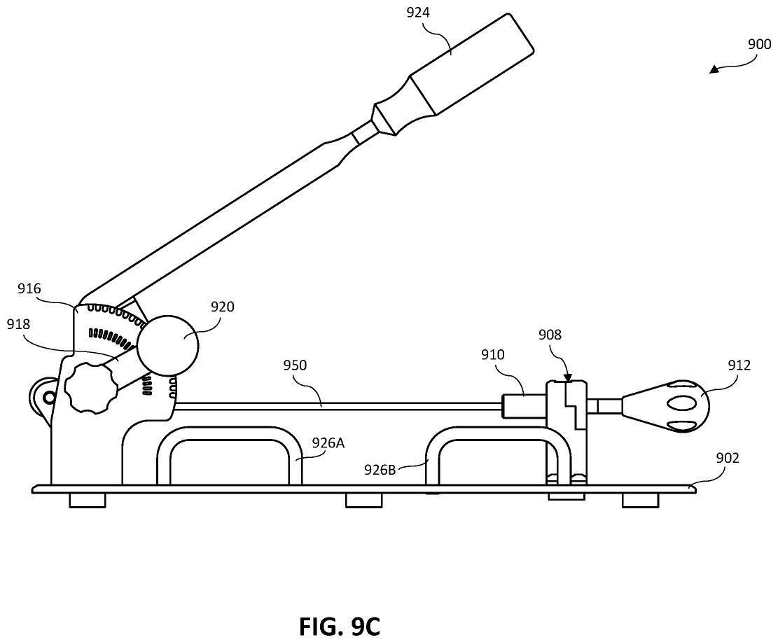

In another instance, the bending tool 106 may be a stationary bending tool that enables hand actuated bends. A spinal rod may be coupled to the stationary bending tool in some aspects. In such instances, the stationary bending tool is adjustable to alter a bend that the stationary bending tool will effect on a spinal rod upon being actuated and where on the spinal rod the bend will be effected. The surgeon may adjust the stationary bending tool to prepare for a bend and then actuate the stationary bending tool to effect the bend on the spinal rod. An example of such a stationing bending tool 106 is presented as the bending tool 900 in FIGS. 9A to 9C described below.

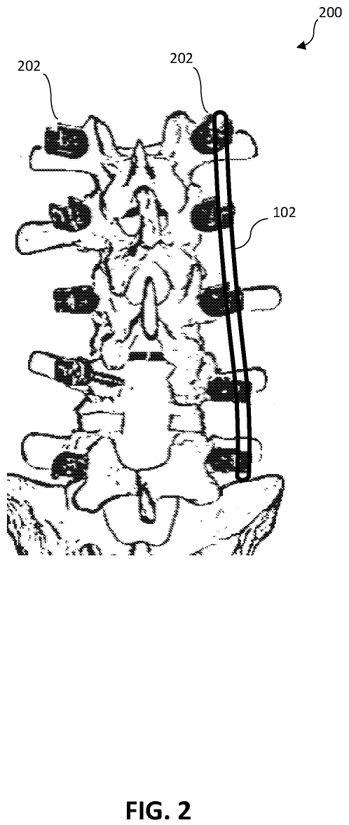

The following description of FIGS. 2 to 9C help to illustrate an example spinal rod preparation method of the present disclosure that may be performed intraoperatively during an invasive spinal rod fusion surgery. Although the example method in connection with FIGS. 2 to 9C is described in a particular order in certain instances, it will be appreciated that many other methods of performing the acts associated with the method may be used. For example, the order of some method elements may be changed, certain elements may be combined with other elements, and some of the elements described may be optional. FIG. 2 illustrates a portion of a vertebral column 200 of a patient (e.g., between L3 and S5). Multiple screws 202 (e.g., pedicle screws) may be installed in the patient during a spinal rod fusion surgery. As shown, the surgeon may shape a flexible rod 102 so that it aligns with or conforms to a path or contour formed by at least some of the screws 202. The flexible rod 102 may be positioned within screw heads of the screws 202. Shaping the flexible rod 102 in this way helps account for the anatomical environment surrounding the screws 202, such as between two adjacent screws 202, that may affect a shape of a final spinal rod construct and that is not accounted for when merely considering the locations of the screws 202.

In an example, accounting for the anatomical environment surrounding the screws 202 may be advantageous in a case in which there is a bony structure surrounding a head of a screw 202 that cannot be easily removed. In such a case, the final spinal rod construct will need to be contoured in a way so that it contours around the bony structure, since it cannot go through the bony structure. The contouring around the bony structure would not be captured by only considering the locations of the heads of the screws 202. Conversely, the flexible rod 102 can be manipulated such that it accounts for the bony structure, which will be captured when forming the final spinal rod construct.

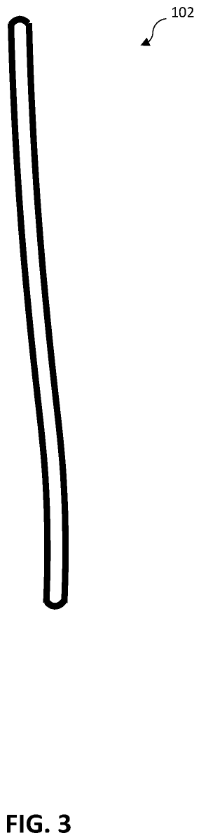

In this example, once the surgeon is satisfied with the shape of the flexible rod 102 relative to the screws 202, the surgeon may remove the flexible rod 102 from the patient. As described above, the flexible rod 102 is constructed such that it maintains its shape after removal. FIG. 3 illustrates the flexible rod 102 having the same shape as in FIG. 2 when it was shaped to conform to the path or contour of the screws 202.

In this example, the surgeon may use a fixture 104 to capture images of the flexible rod 102, such as the example fixture 400 illustrated in FIG. 4. The example fixture 400 is constructed as a platform that includes discrete, fixed positions. In various aspects, the fixture 400 includes a base 402. The base 402 may include a stand 404 configured to maintain a positioning of the flexible rod 102. For example, an end of the flexible rod 102 may be positioned within an opening 406 of the stand 404. In at least some aspects, at least a portion 414 of the stand 404 that includes the opening 406 rotates relative to the base 402. In various aspects, the stand 404 may be configured such that the rotatable portion 414 of the stand 404 rotates to at least two discrete, fixed positions. For example, in the illustrated aspect, the stand 404 includes a notch 408 forming an end stop 410A and an end stop 410B, and an arm 412 that slides within the notch 408. The arm 412 may be connected to or integral with the rotatable portion 414 of the stand 404. When the arm 412 is in contact with the end stop 410A, the rotatable portion 414 of the stand 404 is in a first discrete, fixed position (e.g., a sagittal position). When the arm 412 is in contact with the end stop 410B, the rotatable portion 414 of the stand 404 is in a second discrete, fixed position (e.g., a coronal position). In at least some aspects, the end stops 410A and 410B are a ninety degree rotation of the rotatable portion 414 away from one another.

In other aspects, the fixture may include other mechanisms for enabling discrete, fixed positions. For example, the base 402 may include a magnet having a polarity (e.g., north) at each of a first position and a second position. In such examples, the arm 412 may include a magnet having the opposite polarity (e.g., south) that interfaces with the magnet of the base 402 to maintain the arm 412 in a fixed, discrete position. In another example, the base 402 may include a notch or well at each of a first position and a second position. In such examples, the arm 412 may include a spring pin that engages the notches or wells to maintain the arm 412 in a fixed, discrete position.

In some aspects, the arm 412 may include a fiducial marker 506 (FIG. 5). In such aspects, the fiducial marker 506 helps the vision of a computing system (e.g., the bending instructions generation subsystem 110) detect which discrete, fixed position the rotatable portion 414 is in. In some aspects, the base 402 of the fixture 400 may include a guide 418 for placement of a fiducial card 502 (FIG. 5). In such aspects, the fiducial card 502 helps create a point of reference between real space and image space for the vision (e.g., the image capture device 116) of the bending instructions generation subsystem 110.

In some aspects, the base 402 may include the guides 420A and 420B for the placement of a backboard 504 (FIG. 5). The backboard 504 provides a consistent backdrop for the vision of the bending instructions generation subsystem 110, which helps with accuracy of the digital three-dimensional representation 702 generation. In some aspects, the base 402 may include the guides 422A, 422B, and 422C for the placement of a larger backboard (not illustrated) than the backboard 504, which may be beneficial in certain instances. For example, typically the backboard 504 is large enough to provide a consistent backdrop behind the entirety of the flexible rod 102; however, in certain instances the flexible rod 102 may have a large bend that requires a wider backboard. The backboard 504 and the larger backboard may be constructed of any suitable material with a consistent coloring (e.g., white).

Continuing with the example spinal rod preparation procedure for the invasive spinal rod fusion surgery, the surgeon may mate the shaped flexible rod 102 to the fixture 400, such as by inserting an end of the shaped flexible rod 102 within the opening 406, as FIG. 5 illustrates. In some aspects, an adapter may be placed on the end of the shaped flexible rod 102 that conforms to the opening 406. The adapter uniformly engages the opening 406 to maintain positioning of the shaped flexible rod 102 mated to the fixture 400. In some aspects, the surgeon may indicate on the bending instructions generation subsystem 110 that an invasive procedure is occurring (e.g., by selecting an option on the user interface).

In this example, the surgeon may capture two orthogonal images of shaped flexible rod 102 using the bending instructions generation subsystem 110. A digital three-dimensional representation 702 of the shaped flexible rod 102 is generated based on the two captured orthogonal images. FIG. 5 illustrates an example image configuration 500 of the shaped flexible rod 102 in a first position (e.g., a sagittal position). The surgeon may capture a first image of the shaped flexible rod 102 in the image configuration 500. In this first position, the arm 412 of the fixture 400 is in contact with the end stop 410A, and the fiducial marker 506 on the arm 412 is visible in the image configuration 500. The fiducial marker 506 being visible to the bending instructions generation subsystem 110 in the image configuration 500 indicates to the bending instructions generation subsystem 110 that the image configuration 500 is of the shaped flexible rod 102 in the first position. The fiducial card 502 included in the image configuration 500 creates a point of reference between real space and image space for the bending instructions generation subsystem 110. The shaped flexible rod 102 is positioned entirely in front of the backboard 504 in the image configuration 500, which helps ensure accuracy of the digital reconstruction of the shaped flexible rod 102.

The surgeon may then rotate the rotatable portion 414 of the fixture 400 to position the shaped flexible rod 102 in a second position. FIG. 6 illustrates an example image configuration 600 of the shaped flexible rod 102 in the second position (e.g., a coronal position). The surgeon may capture a second image of the shaped flexible rod 102 in the image configuration 600. The shaped flexible rod 102 is rotated ninety degrees in the second position relative to the first position. In the image configuration 600, the arm 412 of the fixture 400 is in contact with the end stop 410B, and the fiducial marker 506 is not visible to the image capture device 116 when an image is captured from the same vantage point as the image captured of the image configuration 500 (e.g., a straight-on front view shown in FIG. 5). The lack of the fiducial marker 506 in an image captured of the image configuration 600 indicates to the bending instructions generation subsystem 110 that the image configuration 600 is of the shaped flexible rod 102 in the second position. The fiducial card 502 included in the image configuration 600 creates a point of reference between real space and image space for the bending instructions generation subsystem 110. The shaped flexible rod 102 is positioned entirely in front of the backboard 504 in the image configuration 600, which helps ensure accuracy of the digital reconstruction of the shaped flexible rod 102. In other examples, the image configuration 600 may be captured prior to capturing the image configuration 500.

The bending instructions generation subsystem 110 may then generate the digital three-dimensional representation 702 of the shaped flexible rod 102 based on the image configuration 500 and the image configuration 600. For example, the bending instructions generation subsystem 110 may segment the flexible rod 102 from the backboard 504. In this example, the flexible rod 102 may be reduced to a point cloud, though other methods may be utilized as described above. The bending instructions generation subsystem 110, in at least some aspects, may map the two-dimensional coordinates of the flexible rod 102 to three-dimensional coordinates to generate the digital three-dimensional representation 702. In at least some aspects, the digital three-dimensional representation 702 may be displayed as part of a GUI on the display 118 of the bending instructions generation subsystem 110. FIG. 7 illustrates an example GUI 700 that may be displayed on the display 118 and with which the surgeon may interact. The GUI 700 visualizes the digital three-dimensional representation 702 for the surgeon. In at least some aspects, the digital three-dimensional representation 702 may include multiple bend points 712. In at least some aspects, the GUI 700 enables the surgeon to three-dimensionally rotate the digital three-dimensional representation 702, which may help the surgeon appreciate different perspectives of the shape of the digital three-dimensional representation 702.

In some instances, the shape of the flexible rod 102 to conform to the path or contour of the screws 202, and therefore the shape of digital three-dimensional representation 702, might not be the surgeon's desired shape for a final spinal rod construct. For example, as a patient lays on a surgical table, the patient's spinal column might be contoured in a less than desired way as compared to the patient's spinal column contour during normal daily activities, and therefore the flexible rod 102 captures a close, but less than desired, spinal column contour. In another example, the surgeon may desire to effect a different curvature than the path or contour of the screws 202 and captured by the flexible rod 102. Stated differently, the surgeon may be willing to tolerate generating some stresses in the flexible rod 102 and/or the screws 202 in order to effect a particular curvature for the patient's spine. The GUI 700, in at least some aspects, enables the surgeon to adjust the shape of the digital three-dimensional representation 702 in such instances. Enabling a surgeon to make adjustments to the digital three-dimensional representation 702 prior to preparing a final spinal rod construct can help eliminate or reduce operator-induced error.

For example, the surgeon may interact with (e.g., directly on the display 118 with the surgeon's finger(s)) an adjustment bar 704 to adjust various attributes of the digital three-dimensional representation 702 (e.g., sagittal or coronal corrections). In some aspects, the surgeon may adjust one or more of a cranial overhang (e.g., mm), a caudal overhang (e.g., mm), an amount of lordosis (e.g., degrees), an amount of kyphosis (e.g., degrees), coronal straightening in the coronal plane (unit-less), a quantity of bends, or other suitable attributes of the digital three-dimensional representation 702. A cranial or caudal overhang will be appreciated as added length to the digital three-dimensional representation 702 at its cranial or caudal ends, respectively. In an example, the surgeon may touch a minus button 706 or a plus button 708 to respectively decrease or increase an amount of an attribute.

In some aspects, the surgeon may adjust an attribute of the digital three-dimensional representation 702 as a whole. In some aspects, the surgeon may select one or more distinct points on the digital three-dimensional representation 702 (e.g., by touching distinct point(s)) and may adjust an attribute of the digital three-dimensional representation 702 at a selected point or between selected points. For example, the surgeon may adjust an amount of bend (e.g., an amount of lordosis or kyphosis in the sagittal plane) at a selected point or between selected points. In some aspects, the surgeon may select one or more distinct points and adjust another suitable attribute in relation to the selected point(s). In some examples, by selecting different sets of points, a curve on the digital three-dimensional representation 702 can be modified via linear, exponential or other suitable kernel function approaches.

Once the surgeon is satisfied with the adjustments made to the digital three-dimensional representation 702, the surgeon may initiate the generation of bending instructions, for example, by touching an instruction generation box 710. In at least some aspects, the surgeon may input a material and/or diameter for the final spinal rod into the bending instructions generation subsystem 110 (e.g., via the GUI 700). The material and/or diameter for the final spinal rod determine, at least in part, the amount of spring-back that the final spinal rod will have, which is used to calibrate the generated bend instructions with a bending tool. FIG. 8 illustrates an example GUI 800 including example bending instructions 802 generated by the bending instructions generation subsystem 110. The bending instructions 802 indicate how a spinal rod should be bent using a suitable bending tool. In this example, the bending instructions 802 provide values or instructions for a surgeon to bend a spinal rod using the example bending tool 900 shown in FIGS. 9A to 9C. The example bending tool 900 will now be described, and then the bending instructions 802 will be described in view of the bending tool 900.

FIG. 9A illustrates a perspective view of an example bending tool 900. FIGS. 9B and 9C illustrate a top view and side view, respectively, of the example bending tool 900 having a loaded spinal rod 950. The example bending tool 900 includes a base 902. In some aspects the base 902 may include a length measurement track 904. A scale 906 of measurement indicators (e.g., mm) may be next to the length measurement track 904. A surgeon may position a spinal rod 950 within the length measurement track 904 in order to measure a length of at least a portion of the spinal rod 950. For instance, the surgeon may use the measured length to cut the spinal rod 950 to a desired length. In at least some aspects, the bending tool 900 may include a track 914. A rod holder 908 may be configured to slide translationally within the track 914 in the direction of the arrow 934. In at least some aspects, a scale of indicators (e.g., numerals, letter, quantities, etc.) may be positioned along the track 914. The rod holder 908 is configured to hold a spinal rod 950. For example, the rod holder 908 may include a support 910 having an opening configured to accept the spinal rod 950. In at least some aspects, rotating a knob 912 (e.g., about the axis 930 in either direction of the double-sided arrow 932) rotates the support 910, and thus rotates a spinal rod 950 loaded into the support 910. In some instances, the rod holder 908 may include a scale that indicates a rotational position of the support 910 and the knob 912.

In at least some aspects, the example bending tool 900 may include an actuator arm 924 configured to rotate about an axis in the direction of the arrow 936. For example, the actuator arm 924 may be translated in one direction (e.g., away from the rod holder 908) to place the bending tool 900 in a pre-actuation position, and may be translated in the opposite direction (e.g., towards the rod holder 908) to actuate the arm and effect a bend on a spinal rod 950. In at least some aspects, the actuator arm 924 includes a bender end 946 that rotates with the rest of the actuator arm 924. In some instances, the bender end 946 includes a guide 942. A spinal rod 950 may rest within the guide 942. In some aspects, the bending tool 900 may include a guide 944 aligned with the guide 942, such that the long axes of the guides 942 and 944 are in the same plane. In some aspects, when the spinal rod 950 is positioned within the guide 942 and/or the guide 944, at least the guide 942 or the guide 944 provides resistance that helps maintain the spinal rod 950 within the guide 942 or 944 providing resistance. For example, the guide 942 and/or 944 providing resistance may include spring-like biased ends that expand apart to allow entry of the spinal rod 950 and contract towards one another to help maintain the spinal rod 950 from exiting. The guide 944 does not rotate with the actuator arm 924. As the actuator arm 924 is actuated, an angle formed by the guide 942 and the guide 944 changes, which when a spinal rod 950 is positioned within the guides 942 and 944, effects a bend in the spinal rod 950.

In some aspects, the bending tool 900 may include a knob 922. The knob 922 may rotate the guide 944 to adjust its angle. The long axes of the guides 942 and 944 remain within a same plane as the guide 944 is rotated. Adjusting the angle of the guide 944 adjusts a bend radius between cervical and lumbar spinal rods. Adjusting the angle of the guide 944 also moves the bend axis for both the cervical and lumbar spinal rod cases.

In at least some aspects, the bending tool 900 may include an angle base 916. A position of an arm 918 may be adjustable relative to the angle base 916. For example, the angle base 916 may include a set of notches 926, and a shaft 928 extending through the arm 918 may be adjustably positioned within a respective notch 926. In various aspects, the shaft 928 may be spring-biased. For example, a surgeon may pull a knob 920 in the direction of the arrow 937 to remove the shaft 928 from within a notch 926. The surgeon may then slide the arm 918 along the angle base 916 to a desired position and release the knob 920 causing the spring bias to advance the shaft 928 in the direction of the arrow 939 into a respective notch 926. Adjusting the position of the arm 918 sets an amount of bend that the bending tool 900 will effect upon actuation of the actuator arm 924. In some aspects, the angle base 916 may include a scale 938 with indicators (e.g., numerals, letters, quantities, symbols, etc.) for each respective notch 926, which helps indicate an amount of bend that the bending tool 900 is set to effect.

In this example, the arm 918 includes a stop block 940. Adjusting the position of the arm 918 also adjusts the position of the stop block 940. As the actuator arm 924 is actuated (e.g., translated in the direction of the arrow 936 towards the rod holder 908), the actuator arm 924 can be translated only so far as the stop block 940. Stated differently, the actuator arm 924 contacts the stop block 940 and is prevented from translating further. In this way, adjusting a position of the arm 918 and the stop block 940 sets a bend angle that the actuator arm 924 is able to effect.

In some aspects, the example bending tool 900 may include a handle 926A and/or a handle 926B. The handle 926A and/or the handle 926B may help provide for easy handling of the bending tool 900. For instance, a surgeon or other individual may grab the handles 926A and 926B to transport the bending tool 900 from one location to another.

Returning now to the discussion of the example bending instructions 802 in view of the example bending tool 900, the example bending instructions 802 may include a series of steps related to actions to be performed with the bending tool 900. In this example, a first instruction may designate a specific length of spinal rod to be selected (e.g., 248 mm). In some instances, a surgeon may need to cut a spinal rod 950 down to the designated length. For example, a surgeon may position a 275 mm length spinal rod 950 within the length measurement track 904, indicate a 248 mm position on the spinal rod 905 using the scale 906, and cut the spinal rod 950 at the indicated 248 mm position. In some aspects, a second instruction may be to load the spinal rod 950 into the bending tool 900 (e.g., into the support 910). In some instances, a cranial end of the spinal rod 950 may be loaded into the support 910.

In this example, the bending instructions 802 include instructions for four bends to be performed that relate to the bending tool 900. In other examples, the bending instructions 802 may include instructions for other suitable quantities (e.g., two, three, five, etc.) of bends depending on a patient. In view of the bending tool 900, the instructions for each bend include a value for translating or advancing the rod holder 908, rotating the knob 912, and setting a position of the arm 918 to set a bend angle. For example, the instructions for a bend “1” direct a surgeon to translate the rod holder 908 to a value of “6” on the scale along the track 914, rotate the knob 912 to a value of “0” on the scale of the rod holder 908, and set the position of the arm 918 to a value of “C” on the scale 938. Once the surgeon positions the bending tool 900 according to these instructions, the surgeon may actuate the actuator arm 924 to effect the first bend on the spinal rod 950.

In this example, the instructions for a bend “2” then instruct the surgeon to translate the rod holder 908 to a value of “11” on the scale along the track 914, rotate the knob 912 to a value of “100” on the scale of the rod holder 908, and set the position of the arm 918 to a value of “B” on the scale 938. Once the surgeon positions the bending tool 900 according to these instructions, the surgeon may actuate the actuator arm 924 to effect the second bend on the spinal rod 950. The surgeon may then follow the instructions for the bend “3” and the bend “4” to effect the third and fourth bends on the spinal rod 950. It should be appreciated that the spinal rod 950 need not be removed from the bending tool 900 between effecting the different bends. After effecting all of the bends on the spinal rod 950 in the bending instructions 802, the spinal rod 950 may be final and prepared for the surgeon to install in the patient.

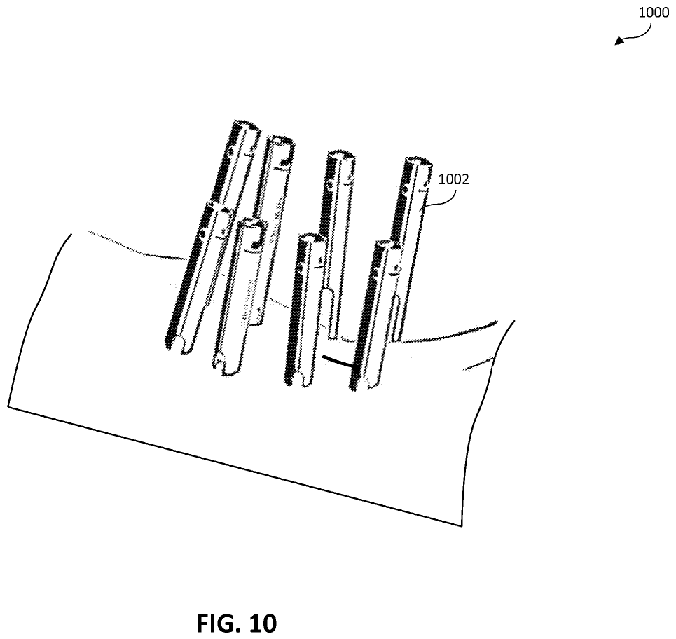

The following description of FIGS. 10 to 14 help to illustrate an example spinal rod preparation method of the present disclosure that may be performed intraoperatively during a minimally invasive spinal rod fusion surgery. Although the example method in connection with FIGS. 10 to 14 is described in a particular order in certain instances, it will be appreciated that many other methods of performing the acts associated with the method may be used. For example, the order of some method elements may be changed, certain elements may be combined with other elements, and some of the elements described may be optional. FIG. 10 illustrates a patient 1000 having installed minimally invasive surgery (MIS) towers 1002. It will be appreciated that only one MIS tower 1002 is indicated in FIG. 10 solely for the sake of clarity. The MIS towers 1002 may be any suitable MIS tower known in the art. The MIS towers 1002 are inserted through the skin 1002 of the patient 1000 and are in contact with screws (e.g., the screws 202) installed on the vertebrae of the patient 1000.

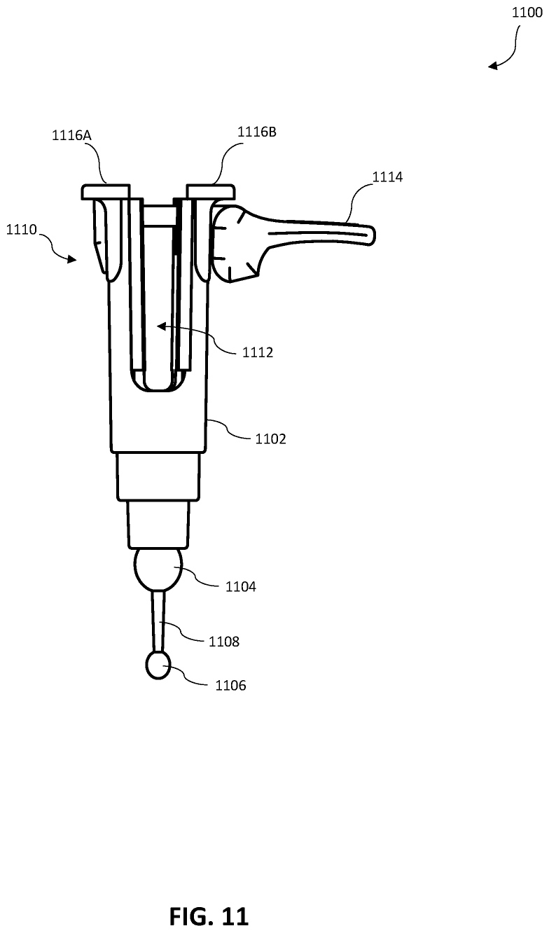

In this minimally invasive example, the surgeon may utilize a set of pointers to generate a digital three-dimensional representation (e.g., the digital three-dimensional representation 702) of an intended spinal rod. FIG. 11 illustrates an example pointer 1100. The example pointer 1100 includes a body portion 1102. The body portion 1102 may be constructed such that at least a portion of the body portion 1102 may be positioned within a MIS tower 1002 in various examples. In at least some aspects, the pointer 1100 includes a fiducial marker 1104 and a fiducial marker 1106 connected by a shaft 1108. In some instances, the shaft 1108 may be straight as illustrated. In at least some instances, the shaft 1108 may be rigid. In at least some aspects, the body portion 1102 includes a clip 1110 configured to attach the pointer 1100 to an object placed through the clip opening 1112. A handle 1114 of the clip 1110 is illustrated in an example open position. Actuating the handle 1114 towards the body portion 1102 causes a first member 1116A and a second member 1116B of the clip 1110 to move towards one another, thus applying compressive force to an object placed though the clip opening 1112.

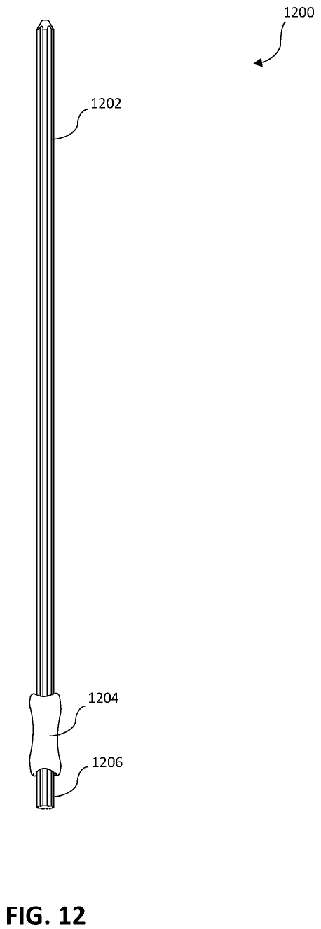

In various aspects, one or more pointers 1100 may be attached to a reference structure so that multiple pointers 1100 may be analyzed with respect to one another. For example, the reference structure may be an example scan rod 1200 shown in FIG. 12. The example scan rod 1200 includes a rod 1202, which in at least some instances is rigid. When the one or more pointers 1100 are attached to the scan rod 1200, the rod 1202 is positioned through the respective clip openings 1112 of the pointers 1100. In at least some aspects, the example scan rod 1200 includes a handle 1204 at an end of the rod 1202. A surgeon may hold the handle 1204 while using the scan rod 1200. In at least some aspects, the scan rod 1200 may include a mating protrusion 1206. The mating protrusion 1206 may be configured to mate the scan rod 1200 with a fixture 104 (e.g., with the opening 406 of the fixture 400) for image capture.

In other examples, the pointer 1100 may have other suitable constructions. For example, the pointer 1100 may alternatively be non-rigid. In such examples, a non-rigid pointer 1100 may be used with a non-rigid or malleable reference structure (e.g., similar to the flexible rod 102) that a surgeon could use and deform to map outpatient spinal contour both within and outside of the surgical space. An advantage of a rigid reference structure, such as scan rod 1200, is that it allows for pointers 1100 to be attached at varying angles of incidence, though a non-rigid reference structure may be used.

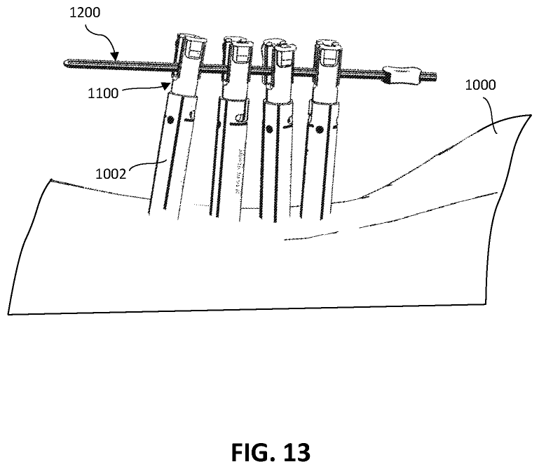

Continuing with the example spinal rod preparation procedure for the minimally invasive spinal rod fusion surgery, a surgeon may position pointers 1100 within at least some of the MIS towers 1002 installed in the patient 1000, as shown in FIG. 13. The surgeon may attach each of the pointers 1100 to the scan rod 1200. In some aspects, images or video of the pointers 1100 attached to the scan rod 1200 may be captured in any of the suitable manners described above in connection with the invasive spinal rod preparation procedure. In some examples, the images or video may be captured in situ while the pointers 1100 are attached to the scan rod 1200 and remain at or within the MIS towers 1002. For example, the MIS towers 1002 may have openings that enable an image capture device (e.g., the image capture device 116) to capture the respective fiducial markers 1104 and 1106 of the pointers 1100 when the pointers 1100 are positioned within the MIS towers 1002. In some examples, a three-dimensional scanner may be utilized to map an intended spinal rod contour that creates a three-dimensional representation of the environment surrounding the pointers 1100. In some examples, the pointers 1100 attached to the scan rod 1200 may be used outside of the surgical space to map points along x-rays, CT scans, etc.

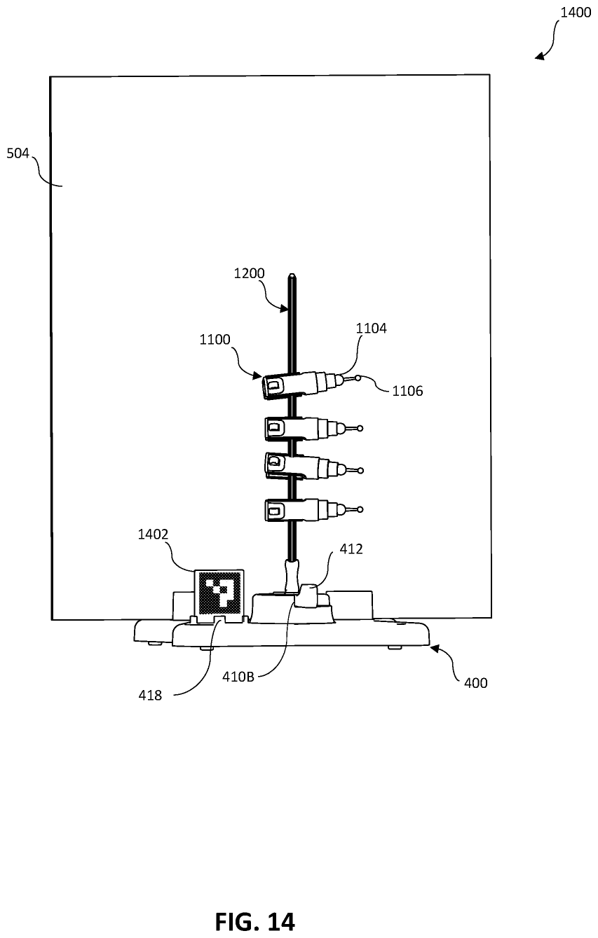

In this example, the surgeon may remove the pointers 1100 attached to the scan rod 1200 from the MIS towers 1002 for image or video capture. As illustrated in the image configuration 1400 of FIG. 14, the surgeon may mate the scan rod 1200 to the fixture 400. In other examples, the surgeon may mate the scan rod 1200 to another suitable fixture 104. In this example, a fiducial card 1402 different than the fiducial card 502 may be positioned in the guide 418 of the fixture 400 during image capture. The fiducial card 1402 may serve a similar purpose to the fiducial card 502. For instance, the presence of the fiducial card 1402 in the captured images creates a point of reference between real space and image space for the bending instructions generation subsystem 110. In some aspects, a surgeon may indicate on the bending instructions generation subsystem 110 that a minimally invasive procedure is occurring (e.g., by selecting an option on the user interface). In some aspects, the surgeon may indicate on the bending instructions generation subsystem 110 how many pointers 1100 are attached to the scan rod 1200. The surgeon may capture a first image of the pointers 1100 attached to the scan rod 1200 in a first position with the fiducial marker 506 visible to the bending instructions generation subsystem 110. And the surgeon may capture a second image of the pointers 1100 attached to the scan rod 1200 in a second position with the fiducial marker 506 not being visible to the bending instructions generation subsystem 110, so that the first and second positions are rotated ninety degrees relative to one another.

Based on the captured images or video of the pointers 1100 attached to the scan rod 1200, the bending instructions generation subsystem 110 may generate a digital three-dimensional representation of an intended spinal rod (e.g., the digital three-dimensional representation 702). For instance, in this example, the bending instructions generation subsystem 110 may segment the fiducial markers 1104 and 1106 of each of the respective pointers 1100 from the backboard 504 in each of the images. The two-dimensional coordinates of the fiducial markers 1104 and 1106 from the two orthogonal images may be mapped to three-dimensional coordinates. In various aspects, the bending instructions generation subsystem 110 may determine directional vectors from the fiducial markers 1104 and 1106 of each of the respective pointers 1100. Each directional vector may be determined as a straight line from the three-dimensional coordinate of the fiducial marker 1104 to the three-dimensional coordinate of the fiducial marker 1106 of a respective pointer 1100. A directional vector indicates a direction that a MIS tower 1002, within which a respective pointer 1100 is or was inserted, is pointing. Based on the directional vectors and known dimensions of the MIS towers 1002 and pointers 1100, locations (e.g., coordinates) in three-dimensional space of the ends of the MIS towers 1002 that are within the patient may be determined. Using the determined locations of the ends of the MIS towers 1002 within the patient, the bending instructions generation subsystem 110 generates the digital three-dimensional representation 702 of an intended spinal rod. In this way, the set of pointers 1100 enable determining an intended spinal rod contour without relying upon capturing the specific location or coordinates in three-dimensional space of the screws 202 installed in the patient.

The pointers 1100 account for surrounding anatomical structures by following trajectories provided by the MIS towers 1002. As described, the pointers 1100 help determine locations of the ends of the MIS towers 1002, and therefore the pointers 1100 account for anatomy because the purpose of the MIS towers 1002 is to give access to the anatomy, which indirectly accounts for anatomy. Furthermore, an additional advantage of the pointers 1100 versus typical systems is that multiple trajectories of each of the pointers 1100 are captured all at once, capturing the contour of multiple areas of the spinal column at once. Conversely, a typical system captures spinal contour at different areas one by one.

The remaining aspects of the example spinal rod preparation procedure for the minimally invasive spinal rod fusion surgery may be similar to that described above for the invasive surgery after the digital three-dimensional representation 702 is generated. For instance, the surgeon may view, and in some instances adjust, the digital three-dimensional representation 702 via a graphical user interface (e.g., the GUI 700 and the GUI 800). The surgeon may then initiate the generation of bending instructions 802. Following the generated bending instructions, the surgeon may bend a surgical rod at one or more locations on the surgical rod, for example, using the bending tool 900. Once the surgical rod is bent, the surgeon may install the final spinal rod construct into the patient.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A spinal rod preparation system comprising:
a flexible rod configured to be manipulated to a bent state;
a bending instructions generation subsystem including:
an image capture device;
a memory; and
a processor in communication with the memory, the processor configured to:
receive at least two images of the flexible rod in the bent state captured by the image capture device,
generate a digital three-dimensional representation of the flexible rod in the bent state based on the at least two received images,
receive adjustment information that adjusts the digital three-dimensional representation of the flexible rod to an adjusted shape, and
generate bending instructions for bending a spinal rod so that it substantially conforms to the adjusted shape of the digital three-dimensional representation; and
a bending tool configured to enable a user to bend a straight spinal rod according to the generated bending instructions.

2. The spinal rod preparation system of claim 1, further comprising a fixture configured to maintain a positioning of the flexible rod while the image capture device captures the at least two images of the flexible rod in the bent state.

3. The spinal rod preparation system of claim 2, wherein the flexible rod extends within a plane perpendicular to a base of the fixture when maintained by the fixture.

4. The spinal rod preparation system of claim 2, wherein the fixture includes a fiducial marker, wherein the fiducial marker is captured in the at least two images of the flexible rod.

5. The spinal rod preparation system of claim 1, wherein the flexible rod is constructed of a malleable material.

6. The spinal rod preparation system of claim 1, wherein the flexible rod includes a plurality of articulating joints.

7. The spinal rod preparation system of claim 1, wherein the flexible rod is constructed of a material capable of transitioning from malleable to rigid upon a transitioning event.

8. The spinal rod preparation system of claim 1, wherein the processor is configured to generate the digital three-dimensional representation based on three or more images via a photogrammetry method.

9. The spinal rod preparation system of claim 1, wherein the image capture device is configured to capture video, and wherein the processor is configured to generate the digital three-dimensional representation based on a captured video of the flexible rod in the bent state.

10. A spinal rod preparation method comprising:
aligning a flexible rod with a plurality of screws installed in a patient, wherein aligning the flexible rod includes bending the flexible rod to conform to a path of the plurality of screws;
capturing at least two images of the aligned flexible rod via an image capture device;
generating a digital three-dimensional representation of the aligned flexible rod via a bending instructions generation subsystem based on the at least two captured images;
adjusting the digital three-dimensional representation of the aligned flexible rod via a user interface of the bending instructions generation subsystem; and
bending a spinal rod using a bending tool according to bending instructions generated by the bending instructions generation subsystem based on the adjusted digital three-dimensional representation.

11. The spinal rod preparation method of claim 10, wherein the images are captured while the flexible rod is positioned within heads of the plurality of screws.

12. The spinal rod preparation method of claim 10, wherein the images are captured using a fixture away from the plurality of screws.

13. The spinal rod preparation method of claim 10, wherein capturing the at least two images includes capturing a first image of the aligned flexible rod in a first position and a second image of the aligned flexible rod in a second position, and wherein the first position is orthogonal to the second position.

14. The spinal rod preparation method of claim 10, wherein adjusting the digital three-dimensional representation includes interacting with a graphical user interface including the digital three-dimensional representation.

15. The spinal rod preparation method of claim 14, wherein adjusting the digital three-dimensional representation includes selecting one or more points on the digital three-dimensional representation and adjusting an amount of curvature at or between the one or more points.

16. A spinal rod preparation system comprising:
a scan rod;
a plurality of pointers each having at least two fiducial markers connected to one another by a shaft, and each being configured to attach to the scan rod;
a bending instructions generation subsystem including:
an image capture device;
a memory; and
a processor in communication with the memory, the processor configured to:
receive at least two images of the plurality of pointers attached to the scan rod captured by the image capture device,
generate a digital three-dimensional representation of a spinal rod based on the at least two received images,
receive adjustment information that adjusts the digital three-dimensional representation to an adjusted shape, and
generate bending instructions for bending a spinal rod so that it substantially conforms to the adjusted shape of the digital three-dimensional representation; and
a bending tool configured to enable a user to couple a straight spinal rod to the bending tool and bend the straight spinal rod according to the generated bending instructions.

17. The spinal rod preparation system of claim 16, wherein generating the digital three-dimensional representation of the spinal rod includes determining a vector of a direction in which a pointer is directed based on the at least two fiducial markers of the pointer and generating the digital three-dimensional representation based on the determined vectors for each of the plurality of pointers.

18. The spinal rod preparation system of claim 16, further comprising a fixture configured to maintain a positioning of the scan rod while the image capture device captures the at least two images of the plurality of pointers attached to the scan rod.

19. The spinal rod preparation system of claim 16, wherein each pointer is configured to be inserted partially within a MIS tower installed in a patient.

20. The spinal rod preparation system of claim 16, wherein the bending tool is configured to adjust a positioning of a coupled spinal rod both translationally and axially, and to adjust a bend angle that the bending tool effects upon the coupled spinal rod in response to an arm of the bending tool being actuated.

21. The spinal rod preparation system of claim 16, wherein the bending instructions include at least one set of instructions for at least one bend, each set of instructions including a set translational position for the coupled spinal rod, a set rotational position for the coupled spinal rod, and a set bend angle.

22. A spinal rod preparation method comprising:
positioning a plurality of pointers into respective MIS towers installed in a patient, each pointer having at least two fiducial markers connected to one another by a shaft;
attaching the plurality of pointers to a scan rod;
capturing at least two images of the plurality of pointers attached to the scan rod via an image capture device;
generating a digital three-dimensional representation of a spinal rod via a bending instructions generation subsystem based on the at least two captured images;
adjusting the digital three-dimensional representation of a spinal rod via a user interface of the bending instructions generation subsystem; and
bending a spinal rod using a bending tool according to bending instructions generated by the bending instructions generation subsystem based on the adjusted digital three-dimensional representation.

23. The spinal rod preparation method of claim 22, wherein the spinal rod is coupled to the bending tool while bending the spinal rod, and wherein bending the spinal rod according to the bending instructions includes:
adjusting the bending tool so that the coupled spinal rod is at a first translational position and a first rotational position,
adjusting the bending tool to a first set bend angle, and
actuating an arm of the bending tool.

24. The spinal rod preparation method of claim 23, wherein bending the spinal rod according to the bending instructions further includes:
adjusting the bending tool so that the coupled spinal rod is at a second translational position and a second rotational position, the second translational position being different than the first translational position,
adjusting the bending tool to a second set bend angle, and
actuating the arm of the bending tool.
